(19) United States
(12) Patent Application Publication
(10) Pub. No.: US 2011/0160523 A1
(43) Pub. Date: Jun. 30, 2011

(54) Method of Training a Living Body to not React to Substances

(76) Inventor: Dawn Nowlin, Santa Fe, NM (US)

(21) Appl. No.: 13/044,986

(22) Filed: Mar. 10, 2011

Related U.S. Application Data
(63) Continuation-in-part of application No. 12/235,360, filed on Sep. 22, 2008, now abandoned.
(60) Provisional application No. 60/974,195, filed on Sep. 21, 2007.

(51) Int. Cl. A61M 21/00; A61N 1/36

(52) U.S. Cl. 600/26; 607/3

(57) ABSTRACT

A method of conditioning a living body of a patient to associate the positive effects of sensory stimulation of the sympathetic ganglia with digital audio representations of allergens or other offending substances to modify errant interpretations of the immune system or other systems involved in a reaction to ultimately cease or reduce negative reactions to such allergens or substances. In addition, a patient's allergies or sensitivities are treated by using digital representations, preferably provided via a computer, as opposed to exposing the patient to the actual substances. Sensory stimulation is used in conjunction with the digital audio signals to condition the body to react more appropriately to the substances without the actual substance being exposed to the patient.

This is a Continuation-In-Part application of non-provisional application No. 12/235,360 filed on Sep. 22, 2008, which in turn claims priority to provisional patent application No. 60/974,195 filed on Sep. 21, 2007, the contents of both of which are hereby incorporated by reference in their entirety.

### FIELD OF THE INVENTION

The present invention is a method of conditioning, a living body of a patient to associate the positive effects of sensory stimulation of the sympathetic ganglia as the primary stimulus with digital audio representations of allergens or other offending substances as the secondary stimulus to modify errant interpretations of the immune system or other systems involved in a reaction to ultimately cease negative reactions to such allergens or substances. In addition, the method of the present invention serves to improve a patient's symptoms caused by allergies or sensitivities by using audio digital representations as opposed to exposing the patient to the actual substances during treatment.

### BACKGROUND OF THE PRESENT INVENTION

Allergies and sensitivities are the result of the immune system or other bodily systems reacting inappropriately to harmless, naturally occurring substances, which can affect virtually every part of the body. These reactions are abnormal. Rather than defending against a pathogen, the immune system misidentifies or misinterprets a harmless substance and reacts inappropriately.

While a “true” allergy is defined by IgE-mediated reactions with immune involvement, a large percentage of these types of conditions do not involve the immune system. Such reactions that do not involve the immune system are referred to as sensitivities. The symptoms of sensitivities are often as pronounced and can even be as severe as those of true allergies, with no immune involvement. It has only been recently recognized that sensitivity-related illness (SRI) may involve various organ systems and evoke wide-ranging physical or neuropsychological manifestations. One or more of the major organ systems can also be involved with a true allergy, in addition to immune involvement, presenting symptoms that correspond to that system.

The inventor has concluded that the body's response to a substance depends on the interpretation or perception of that substance. If the body perceives the substance as harmful, it will react in defense, in the case of immune involvement or with sympathetic hyperactivity, in the case of organ system involvement. If the substance is benign, an inappropriate reaction is the result of an errant perception of the substance leading to allergic reactions or sensitivity reactions.

This invention is not based on treating the immune system, but instead approaches the physiological reaction of allergies and sensitivities by addressing the perceptual error. The invention takes advantage of adaptive mechanisms of the body utilizing a conditioning method that enables the human brain and cognitive processes to adapt to the internal and external milieu. Traditional cognitive studies typically depend on stimulus only; however, this invention utilizes stimulus and context perceived by the subject.

By exposing the person to the offending substance while simultaneously introducing a sensory stimulus, the body associates the positive stimulus with the benign substance and alters its perception, interpreting the substance as beneficial. The inventor has found that the substance being treated may be introduced to the subject as an audio digital representation of the substance provided by a computer or other device. This is achieved by transmitting a representational signal of the offending agent to the surface of the skin. The cognitive processes of the body perceive the signal in a similar or identical fashion as if it were exposed to the actual substance in treatment. The inventor has found that the subconscious and conscious mind can interpret the representation of the substance used in treatment for the conditioned effect.

The first known study on the phenomenon of conditioning was in 1902, by Ivan Pavlov, demonstrating that conditioned reflexes may be learned either by repetitive stimuli or by associating two stimuli. The conditioning process causes a learned behavior that responds to the associated stimulus based on the newly interpreted meaning of that stimulus. Pavlov concluded that a connection was made in the nervous system linking an environmental stimulus to an unconditioned reflex, transforming the reflex into a conditioned reflex, activated by an external stimulus. This invention utilizes sensory stimulus to the sympathetic ganglia as the primary stimulus and contextual audio digital signals of an offending agent as the secondary stimulus to create a new association or interpretation of the treated substance.

The process which Pavlov described as a formation of conditioned reflexes is essentially a translation of messages from a psychological sign system into messages of a system of somatic signs and vice versa. With this translation, a connection develops between the psychological and the somatic levels. The stimulus to the body and use of contextual representations for the mind or cognitive processes can be integrated and, in fact, are connected.

Pavlov concluded that in animals there exists only a first system of signals of reality, allowing the brain to receive and analyze stimuli within the organism as well as outside the organism. In humans, there exists both this first level as well as a second level signals: language or symbols. Words and symbols can function as stimuli in humans, so real and effective, that they can mobilize humans just as a concrete stimulus. Words are symbols, abstractions; the conditioned stimulus can be generalizable.

The digital signal used in treatment is initially created from a textual representation, including a specific sequence of words that allows for a more specific representation of the substance. This textual representation is converted to an audio signal and transmitted repeatedly during treatment. It has also been found that the perceptual process may be controlled at the subconscious level, and as such, the digital audio signals are played at a speed that in unintelligible to the conscious mind in order to engage the unconscious mind. However, for exemplary results, the invention engages both the conscious and unconscious mind by transmitting the digital signals at decibels that are barely audible, as an indicator to the conscious mind of transmission, since the substance represented by the signal is known to the subject prior to treatment. The digital audio signal is transmitted to surface of the skin through an arm cuff.

### SUMMARY OF THE PRESENT INVENTION

The method of the present invention is as follows: While a signal, which represents the substance the body is inappropriately reacting to, is transmitted to the body, preferably via a computer playing a digitized sound, points are stimulated evenly down both sides of the spine at the location of the sympathetic ganglia of the organ systems. Stimulation of these areas have been shown to temporarily improve the afferent and efferent function of the effected organ systems and to alleviate sympathetic hyperactivity. The simultaneous stimulation of sympathetic ganglia with the cognitive perception of the signal teaches or reconditions the body to perceive the substance as beneficial as it becomes associated with the positive stimuli and thereby modifies the physiological reaction. The mechanism is not unlike allergy shots, where the goal is to slowly train the body to accept diluted amounts of the allergen. Over time, the body is conditioned to accept the substance. The present invention introduces the exposure along with the positive stimulus, allowing for a much faster process of accepting the substance and a resolution to the associated reactions.

The mechanism underlying the effect of the treatment is a conditioned association that creates a “coupling of meaning” whereby the substance is “coupled” with the positive effect of the stimulus. The modified perception of the harmless substance alters the behavior of the effected system(s). The therapeutic stimulation used in conjunction with the transmission of digital signals conditions the body to accept a harmless substance. The transmission of digital signals has no effect or therapeutic value to the body. It is only the stimulation in conjunction with exposure to the digital representation that allows for a resolve in the physiological error.

The inventor has discovered that the human body can inappropriately react to a vast number of substances including stimuli, such as heat or sunlight. Therefore, a database, preferably maintained by a computer, is used to include a large number of substances and further breaks the substances down into components and further into components of the components down to the actual protein molecule. This methodology allows for superior precision in addressing the exact substance in which the body is reacting.

A joint study was conducted in Seoul, Korea and Kagoshima, Japan in 2010, authorized by the inventor of the present invention and under the direction of Seung-Ho Yi, Ph. D. of Kyung Hee University in Seoul, Korea. The study was conducted to test the efficacy of utilizing digital representational signals in the conditioning process for the treatment of allergies and sensitivities. A total of 85 cases were treated for a range of allergies or sensitivities to shrimp, crab, cucumber, peach, chrysalis, metals, garlic, pineapple, stone fruit, red wine, peppers, coffee, dust/dust mites and cat dander. Patients received between 1-3 treatments during the study. At least 24 hours of interval was required between treatments for each subject. Subjects ranged in age from 9 to 65 years with 49 females and 36 males, all of Korean of Japanese decent. Inclusions criteria were that subjects must have recently experienced allergic or sensitivity reactions. The reactions were to be clearly observed within a 24 hour period. Reactions were not to be present with avoidance, ruling out additional contributing factors. Exclusion criteria included any symptoms that required long term treatment due to the time limit of the study. No control group was employed. No blind test was executed. However, a blindness in the assessment of outcomes was applied.

To assess the efficacy of the treatment, patients reported improvement on a scale from 0-100% with 0=no improvement and 100%=complete resolution. If the patient reported 100% resolution, no symptoms occurred upon exposure after treatment. Reports were collected as subjects visited the test hospital and challenge tests were completed by subjects following 24 hours after treatment. Neither blood tests nor skin tests were included as the majority of cases involved reactions to foods and it is generally recognized that such tests do not provide high predictive value and often do not correlate with symptom manifestation or severity.

Patient reporting and visible evidence is considered to be more clinically relevant, therefore the most accurate tests for treatment results was to challenge the allergen or offending substance after treatment. Future studies on immunochemistry may include blood test and skin scratch tests to verify any potential effects of the immune system. For the purposes of this study, symptomatic reactions from the effective organ systems were the focus for treatment results.

The results of the study showed that 61% of the cases responded with substantial improvement or complete resolution of symptoms and could experience continued exposure without the previous symptoms. Moderate improvement was reported by 22% of the cases and little or no improvement was reported by 16% of the cases. Considering a generally accepted value of placebo effect of 20% in medical treatments, the study confirmed that the treatment produced a clinically significant result. Three months after treatment 46% of cases who responded to the follow up reported a sustained result, 38% reported either an increase in symptoms or return to the original symptoms and 15% reported an improvement from the initial results. Without further studies, this invention is currently not intended for use on cases of anaphylaxis or life threatening allergies or sensitivities.

Provided herein as part of this application is a compendium of many of the substances and some of the elements thereof, to which people can react as allergens or offending agents, the ways that using a computer, physicians may treat that particular agent, and a schematic for the cuff, which may wrap around the patient's arm or leg or positioned against any aspect of the body and is connected to the computer system which transmits the digital representations.

### DETAILED DESCRIPTION OF THE EMBODIMENT(S) OF THE PRESENT INVENTION

The present invention is a method that serves to treat digital audio representations associated with a specific allergen or substance in order to ultimately condition the body's natural physiology to accept such specific irritant that initially caused the allergic or sensitivity reaction. In one embodiment, sensory stimulation is administered to locations near the sympathetic ganglia of the organ systems, along each side of the spine, with the use of pressure, percussion or laser stimulation. This stimulation is utilized to temporarily improve the afferent and efferent function and alleviate sympathetic hyperactivity in the major organ systems which are overreacting. Meanwhile, the digital representation specific to the substance found to cause the irritation is transmitted to the body. The combination of the cognitive perception of the digital audio signals and the stimulation of the sympathetic ganglia teaches or conditions the body to not react to that particular substance. In other words, the different digital signals, provided via a computer, that represent an allergen or irritant transmitted at the same time as applying a positive stimulus ultimately trains the body to associate the allergen or irritant with positive benefit.

The present invention can proceed as follows. First, a speaker is positioned in the proximity of the patient. It should be noted that the patient also might be referred to as person. In one embodiment, the speaker may be placed on the underside of an arm cuff The arm cuff would then be wrapped around the wrist, leg, or otherwise around an extremity of the patient or positioned on any aspect of the body. The cuff interfaces with a computer via a USB port. The speaker would be facing the body of the patient. Then, a digital audio signal is transmitted toward the body of the patient, preferably via, a computer. The signal being transmitted is representative of what the actual potential allergen or offending agent is purported to be. This means that the sound literally can be words identifying the purported allergen. The sound being transmitted, however, will be unintelligible to the patient, yet barely audible. This is because in one embodiment, the sound is converted into a digital format so as to be emitted in a digital format and audio transmission is accelerated so that it is unintelligible. To accelerate the transmission of the sound/ signal, the media playback code in the computer is set to play back the speed at an accelerated rate to play the sound files at 2.0 or higher. While the signal is transmitted toward the body of the patient, a sensory stimulation is applied to the sympathetic ganglia of the major organ systems utilizing pressure, percussion/vibration or laser stimulation, alongside the vertebrae, Additional stimulation may be applied to specific sympathetic ganglia corresponding to organ systems which present the prevalent symptoms. The stimulation, which causes an alleviation of hyperactivity, along with the representation of the substance has the effect of conditioning the patient's body to perceive the allergen or irritant as a physical positive, which in turn will diminish the inappropriate reaction.

Another embodiment of the present invention is such that a thin foam pad encases a device that is 2 feet in length and 4 inches in width and provides a gentle percussion when electricity has been turned on. This is placed on a treatment table and the patient lies on the pad so that the pad makes contact at the location of the spine, specifically between the spine at the paraspinal muscles of the patient. When activated, the device stimulates the sympathetic ganglia areas with a gentle percussion. The percussion occurs while the sound is transmitted toward the body of the patient, as described above. And thus, it would be preferred that in the pad is the speaker, located at the top of the pad (near where the patient's head rests) that transmits the digital audio signals, so that the barely audible sound may still be heard by the patient to engage the conscious mind in the conditioning. The pad replaces the need for the arm cuff noted above, and the stimulation administered to the sympathetic ganglia by the doctor would not be necessary because of the percussion from the pad. It should be understood that the pad and the cuff are of conventional design.

According to the present invention, it should be understood that the doctor conducts a thorough patient intake to assess what substance or substances are causing the inappropriate reaction. Once the substance for treatment is determined, the doctor affixes the arm cuff to the patient. The signal representing the substance is transmitted to the patient via the software program. At the same time, the doctor applies the sensory stimulation to the sympathetic ganglia to alleviate the hyperactivity of the organ systems. Depending on the substance, the session may require multiple treatments for components.

For example, a sensitivity to wheat may be caused by the body reacting to both gluten and gliadin. Such components have been found to be common, so are included in the treatment.

The present invention could, for example, be targeted at the following substances and their corresponding offending elements:

Cat
Cat Hair
Cat Dander
Cat Saliva
Cat Urine
Cat Protein Molecules
   Fel d 1
   Fel d 2

Red Wine
Sulfites
Tannins

Wheat
Gluten
Gliadin

Beans
Soy Beans
Black Beans

It should be understood that the present invention is a method of training a living body of a patient, such that the following steps would be performed: positioning a speaker in the proximity of the patient; transmitting, via a computer, a series of representational signals from the speaker, each signal of the series of signals representing a corresponding allergen or irritant; and administering the series of representational signals to the patient. The present invention also calls for stimulating the sympathetic ganglia when administering the series of representational signals to the surface of the skin via the speaker. Also, according to the present invention, one would ensure that converting each signal of the series of signals into a digital format is carried out. Further, the present invention calls for storing and matching each signal of the series of signals with the corresponding allergen or offending agent in a computer database. Additionally, according to the present invention, one would place the speaker onto a cuff, the cuff configured to secure to an extremity of the patient. Also, the present invention calls for converting each signal of the series of signals into an unintelligible format, Moreover, the present invention can be viewed as positioning a speaker in the proximity of the patient; transmitting a signal from the speaker toward the body of the patient, the signal matched with a corresponding allergen; and stimulating sympathetic ganglia locations while transmitting the signal from the speaker. Furthermore, the present invention should he viewed as positioning a speaker in the proximity of the patient; transmitting, via a computer, a series of signals from the speaker toward the body of the patient, each signal of the series of signals matched with a corresponding substance; facing the speaker toward the body of the patient; converting each signal of the series of signals into a digital format via a computer; storing and matching each signal of the series of signals with the corresponding substance in a computer database; placing the speaker onto a cuff, the cuff configured to secure to an extremity of the patient or anywhere on the surface of the skin; and converting each signal of the series of signals into an unintelligible format.

I claim:

1. A method of training a living body of a patient, comprising:
positioning a speaker in the proximity of the patient;
transmitting, via a computer, a series of representational signals from the speaker, each signal of the series of signals representing a corresponding allergen or irritant; and
administering the series of representational signals to the patient.

2. The method of claim 1, further comprising stimulating the sympathetic ganglia when administering the series of representational signals to the surface of the skin via the speaker.

3. The method of claim 1, further comprising converting each signal of the series of signals into a digital format.

4. The method of claim 1, further comprising storing and matching each signal of the series of signals with the corresponding allergen or offending agent in a computer database.

5. The method of claim 1, further comprising placing the speaker onto a cuff, the cuff configured to secure to the patient.

6. The method of claim 1, further comprising converting each signal of the series of signals into an unintelligible format.

7. A method of training a living body of a patient, comprising:
positioning a speaker in the proximity of the patient;
transmitting a signal from the speaker toward the body of the patient, the signal matched with a corresponding allergen; and
stimulating sympathetic ganglia locations while transmitting the signal from the speaker.

8. A method of training a living body of a patient, comprising:
positioning a speaker in the proximity of the patient;
transmitting, via a computer, a series of signals from the speaker toward the body of the patient, each signal of the series of signals matched with a corresponding substance;
facing the speaker toward the body of the patient;
converting each signal of the series of signals into a digital format via a computer;
storing and matching each signal of the series of signals with the corresponding substance in a computer database;
placing the speaker onto a cuff, the cuff configured to secure to an extremity of the patient or anywhere on the surface of the skin; and
converting each signal of the series of signals into an unintelligible format.
